(19) United States
(12) Patent Application Publication
(10) Pub. No.: US 2023/0149589 A1
(43) Pub. Date: May 18, 2023

(54) Air Filter Device Incorporating Anti-Microbial Filter Media

(71) Applicant: Kraton Polymers LLC, Houston, TX (US)

(72) Inventors: James D Smith, Houston, TX (US); Vijay Mhetar, Houston, TX (US); Roger Tocchetto, Houston, TX (US)

(73) Assignee: Kraton Polymers LLC, Houston, TX (US)

(21) Appl. No.: 17/995,853

(22) PCT Filed: Apr. 19, 2021
(86) PCT No.: PCT/US2021/070412

§ 371 Date: Oct. 8, 2022

Related U.S. Application Data
(60) Provisional application No. 63/011,576, filed on Apr. 17, 2020.
(60) Provisional application No. 63/019,634, filed on May 4, 2020.
(60) Provisional application No. 63/200,300, filed on Feb. 28, 2021.

(51) Int. Cl. A61L 9/16

(52) U.S. Cl. CPC A61L 9/16; A61L 2101/48

(57) ABSTRACT

An air filter device. The air filter device comprises: a filter medium having an inlet surface for taking in air, and a discharge surface for filtered air. The filter medium having a plurality of air passages for the air to flow therethrough. At least a portion of the inlet surface of the filter medium comprises a sulfonated polymer for killing at least 90% microbes in the air within 120 minutes of contact with the surface of the air passages. The sulfonated polymer is selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer has a degree of sulfonation of at least 10%.

### TECHNICAL FIELD

The disclosure relates to air filter systems. More particularly, the disclosure relates to air filter systems incorporating an anti-microbial filter medium.

### BACKGROUND

With the spread of contagious diseases, such as, COVID-19, it becomes necessary to protect people and prevent the people from coming into contact with diseases spreading microbiological molecules such as, viruses, bacteria, etc. Gas streams often carry particulate materials including microbiological molecules. Viral RNA has reportedly been found on return air grilles, return air ducts, and on heating, ventilation, and air conditioning (HVAC) filters. Common filter media include layered materials containing fibers of substances such as fiberglass, metals, polymers, and ceramics. Filters impregnated with biostat type antimicrobial agents are known in the art, for use in central air conditioning and heating systems, to rid air of fungi, bacteria, viruses, algae, yeasts, and mold. These filters have to be periodically replaced.

It is desirable to have air filter systems containing air filters with antimicrobial, or with self-sterilizing effect and long-lasting efficacy, e.g., of at least 30 days, or 60 days, or 90 days, against pathogens including the COVID-19 virus SARS-CoV-2.

### SUMMARY

In a first aspect, an air filter device is disclosed. The air filter device comprises: a filter medium having an inlet surface for taking in air, and a discharge surface for filtered air, the filter medium having a plurality of air passages for the air to flow therethrough. At least a portion of surface of the air passages is coated with a sulfonated polymer layer having a thickness of at least >1 μm for killing at least 95% microbes in the air within 30 minutes of contact with the sulfonated polymer. The sulfonated polymer layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof, the sulfonated polymer has a degree of sulfonation of at least 10%.

In some aspects, the sulfonated polymer layer comprises at least 50 wt. %, more preferably at least 70 wt. %, even more preferably at least 90 wt. %, yet more preferably at least 95 wt. %, still more preferably at least 98 wt. %, even more preferably at least 99 wt. % and most preferably 100 wt. % (i.e. consists) of one or more of the sulfonated polymers.

In another aspect, a method to prepare an anti-microbial filter device is disclosed. The method comprises a solution comprising sulfonated polymer and a solvent; feeding the sulfonated polymer in solution to a multi-nozzle device or a nozzle-free electrospinning with nozzle free device under influence of a high voltage, generating charged jet streams of sulfonated polymer in solution; depositing the charged jet streams of sulfonated polymer in solution onto a filter mat. The sulfonated polymer solidifies thereby forming a layer of sulfonated polymer on the filter mat, forming a filter medium. The layer of sulfonated polymer has a thickness of less than 400 nm, for killing at least 95% microbes in the air within 30 minutes of contact with the filter medium.

### DETAILED DESCRIPTION

The following terms used the specification have the following meanings:

“MERV” means “Minimum Efficiency Reporting Value.” MERV of a filter describes the size of the holes in the filter that allow air to pass through. The higher the MERV rating, the smaller the holes in the filter, the higher the efficiency. MERV is derived from a test method developed by the American Society of Heating, Refrigerating, and Air Conditioning Engineers (ASHRAE). MERV ranges from 1 to 16, with higher values correspond to more efficient filters. The particle size addressed by MERV scale is 0.3 to 10 μm.

“HEPA” means “”High Efficiency Particulate Air.” HEPA filters are employed to remove submicron size particles from the air. HEPA filters refer to a filter capable of filtering out at least 99.97% of 0.3 μm size particles, as evidenced by a DOP (dispersed oil particulate) test. Particles that are larger or smaller are trapped with higher efficiency, with the worst-case particle size results in worst-case efficiency.

“ULPA” means “Ultra-Low Particulate Air.” ULPA standard requires removal of 99.9995% of particles down to 0.12 μm. ULPA filters are only necessary for specialized applications such as microelectronics manufacturing, medical laboratories, electro-surgical operations, clean rooms, etc.

“Filter” herein refers to a device for filtering particles, e.g., dust, pollen, pathogens, germs, microbes such as mold, etc., from the air passing through it, with an air permeable filter medium or substrate, for use in applications such as HVAC (heating, ventilating, and air conditioning).

“Effective amount” refers to an amount sufficient to alter, destroy, inactivate, and/or neutralize microbes, e.g., an amount sufficient to sterilize and kill microbes in contact with outer surface of the face panel in a face shield.

“Ion Exchange Capacity” or IEC refers to the total active sites or functional groups responsible for ion exchange in a polymer. Generally, a conventional acid-base titration method is used to determine the IEC, see for example International Journal of Hydrogen Energy, Volume 39, Issue 10, Mar. 26, 2014, Pages 5054-5062, “Determination of the ion exchange capacity of anion-selective membrane.” IEC is the inverse of “equivalent weight” or EW, which the weight of the polymer required to provide 1 mole of exchangeable protons.

“Microbes” refers to microorganisms including bacteria, archaea, fungi (yeasts and molds), algae, protozoa, and viruses, with microscopic size.

“Surface pH” refers to the pH on the contact surface of the bio-secure material, that results from surface bound moieties e.g., the coating layer. The surface pH can be measured with commercial surface pH measuring instruments, e.g., SenTix™ Sur-electrode from WTW Scientific-Technical Institute GmbH, Weilheim, Germany.

The disclosure relates to an air filter having a filter medium comprising a protective antimicrobial layer that kills microbes within a predefined duration of contact. The filter medium is coated with, protected with, or constructed to have a layer comprising a self-sterilizing (self-disinfecting) sulfonated polymeric material. The sulfonated polymer is employed for killing at least 95% microbes within a pre-defined duration of contact with the sulfonated polymeric coating. In embodiments, the self-disinfecting material comprises, consists of, or consists essentially of a sulfonated polymer.

Self-sterilizing Material—Sulfonated Polymer: Sulfonated polymer refers to polymers having a sulfonate group, e.g., —SO₃, either in the acid form (e.g., —SO₃H, sulfonic acid) or a salt form (e.g., —SO₃Na). The term “sulfonated polymer” also covers sulfonate containing polymers, e.g., polystyrene sulfonate.

The sulfonated polymer is selected from the group of perfluorosulfonic acid polymers (e.g., sulfonated tetrafluoroethylene), sulfonated polyolefins, sulfonated polyimides, sulfonated polyamides, sulfonated polyester, polystyrene sulfonates, sulfonated block copolymers, sulfonated polyolefins, sulfonated polysulfones such as polyether sulfone, sulfonated polyketones such as polyether ether ketone, sulfonated polyphenylene ethers, and mixtures thereof.

The sulfonated polymer is characterized as being sufficiently or selectively sulfonated to contain from 10-100 mol % sulfonic acid or sulfonate salt functional groups based on the number of monomer units or the block to be sulfonated (“degree of sulfonation”), to kill at least 95% of microbes within 120 minutes of coming into contact with the coating material. In embodiments, the sulfonated polymer has a degree of sulfonation of >25 mol %, or >50 mol %, or <95 mol %, or 25-70 mol %. Degree of sulfonation can be calculated by NMR or ion exchange capacity (IEC).

In embodiments, the sulfonated polymer is a sulfonated tetrafluoroethylene, having a polytetrafluoroethylene (PTFE) backbone; (2) side chains of vinyl ethers (e.g., —O—CF₂—O—CF—O—CF₂—CF₂—) which terminate in sulfonic acid groups in a cluster region.

In embodiments, the sulfonated polymer is a polystyrene sulfonate, examples include potassium polystyrene sulfonate, sodium polystyrene sulfonate, a co-polymer of sodium polystyrene sulfonate and potassium polystyrene sulfonate (e.g., a polystyrene sulfonate copolymer), having a molecular weight of 20,000 to 1,000,000 Daltons, or >25,000 Daltons, or >40,000 Dalton, or >50,000, or >75,000, or >100,000 Daltons, or >400,000 Daltons, or <200,000, or <800,000 Daltons, or up to 1,500,000 Daltons. The polystyrene sulfonate polymers can either be crosslinked or uncrosslinked. In embodiments, the polystyrene sulfonate polymers are uncrosslinked and water soluble.

In embodiments, the sulfonated polymer is a polysulfone, selected from the group of aromatic polysulfones, polyphenylenesulfones, aromatic polyether sulfones, dichlorodiphenoxy sulfones, sulfonated substituted polysulfone polymers, and mixtures thereof. In embodiments, the sulfonated polymer is a sulfonated polyethersulfone copolymer, which can be made with reactants including sulfonate salts such as hydroquinone 2-potassium sulfonate (HPS) with other monomers, e.g., bisphenol A and 4-fluorophenyl sulfone. The degree of sulfonation in the polymer can be controlled with the amount of HPS unit in the polymer backbone.

In embodiments, the sulfonated polymer is a sulfonated polyether ketone. In embodiments, the sulfonated polymer is a sulfonated polyether ketone ketone (SPEKK), obtained by sulfonating a polyether ketone ketone (PEKK). The polyether ketone ketone can be manufactured using diphenyl ether and a benzene dicarbonic acid derivative. The sulfonated PEKK can be available as an alcohol and/or water-soluble product, e.g., for subsequent use to coat the face mask or in spray applications.

In embodiments, the sulfonated polymer is a sulfonated poly(arylene ether) copolymer containing pendant sulfonic acid groups. In embodiments, the sulfonated polymer is a sulfonated poly(2,6-dimethyl-1,4-phenylene oxide), commonly referred to as sulfonated polyphenylene oxide. In embodiments, the sulfonated polymer is a sulfonated poly(4-phenoxybenzoyl-1,4-phenylene) (S-PPBP). In embodiments, the sulfonated polymer is a sulfonated polyphenylene having 2 to 6 pendant sulfonic acid groups per polymer repeat, and characterized as having 0.5 meq (SO₃H)/g of polymer to 5.0 meq (SO₃H)/g polymer, or at least 6 meq/g (SO₃H)/g polymer.

In embodiments, the sulfonated polymer is a sulfonated polyamide, e.g. aliphatic polyamides such nylon-6 and nylon-6,6, partially aromatic polyamides and polyarylamides such as poly(phenyldiamidoterephthalate), provided with sulfonate groups chemically bonded as amine pendant groups to nitrogen atoms in the polymer backbone. The sulfonated polyamide can have a sulfonation level of 20 to up to 100% of the amide group, with the sulfonation throughout the bulk of the polyamide. In embodiments, the sulfonation is limited to a high density of sulfonate groups at the surface, e.g., >10%, >20%, >30%, or >40%, or up to 100% of the sulfonated amide group at the surface (within 50 nm of the surface).

In embodiments, the sulfonated polymer is a sulfonated polyolefin, containing at least 0.1 meq, or >2 meq, or >3 meq, or >5 meq, or 0.1 to 6 meq of sulfonic acid per gram of polyolefin. In embodiments, the sulfonated polymer is a sulfonated polyethylene. The sulfonated polyolefin can be formed by chlorosulfonation of a solid polyolefin obtained by polymerization of an olefin or a mixture of olefins selected from a group consisting of ethylene, propylene, butene-1,4-methylpentene-1, isobutylene, and styrene. The sulfonyl chloride groups can then be hydrolyzed, for example, in an aqueous base such as potassium hydroxide or in a water dimethylsulfoxide (DMF) mixture to form sulfonic acid groups. In embodiment, the sulfonated polyolefin is formed by submerging or passing polyolefin object in any form of powder, fiber, yarn, woven fabric, a film, a preform, etc., through a liquid containing sulfur trioxide (SO₃), a sulfur trioxide precursor (e.g., chlorosulfonic acid, HSO₃Cl), sulfur dioxide (SO₂), or a mixture thereof. In other embodiments, the polyolefin object is brought into contact with a sulfonating gas, e.g., SO₂ or SO₃, or gaseous reactive precursor, or a sulfonation additive that evolves a gas SOₓ at elevated temperature.

The polyolefin precursor to be sulfonated can be, for example, a poly-α-olefin, such as polyethylene, polypropylene, polybutylene, polyisobutylene, ethylene propylene rubber, or a chlorinated polyolefin (e.g., polyvinylchloride, or PVC), or a polydiene, such as polybutadiene (e.g., poly-1,3-butadiene or poly-1,2-butadiene), polyisoprene, dicyclopentadiene, ethylidene norbornene, or vinyl norbornene, or a homogeneous or heterogeneous composite thereof, or a copolymer thereof (e.g., EPDM rubber, i.e., ethylene propylene diene monomer). In embodiments, the polyolefin is selected from low density polyethylene (LDPE), linear low density polyethylene (LLDPE), very low density polyethylene (VLDPE), high density polyethylene (HDPE), medium density polyethylene (MDPE), high molecular weight polyethylene (HMWPE), and ultra-high molecular weight polyethylene (UHMWPE).

In embodiments, the sulfonated polymer is a sulfonated polyimide, e.g., aromatic polyimides in both thermoplastic and thermosetting forms, having excellent chemical stability and high modulus properties. Sulfonated polyimide can be prepared by condensation polymerization of dianhydrides with diamines, wherein one of the monomeric units contains sulfonic acid, sulfonic acid salt, or sulfonic ester group. The polymer can also be prepared by direct sulfonation of aromatic polyimide precursors, using sulfonation agents such as chlorosulfonic acid, sulfur trioxide and sulfur trioxide complexes. In embodiments, the concentration of sulfonic acid groups in the sulfonated polyimide as measured by ion exchange capacity, IEC, varying from 0.1 meq/g to above 3 meq/g, or at least 6 meq/g.

In embodiments, the sulfonated polymer is a sulfonated polyester, formed by directly sulfonating a polyester resin in any form, e.g., fiber, yarn, woven fabric, film, sheet, and the like, with a sulfuric anhydride-containing gas containing sulfuric anhydride, for a concentration of the sulfone group on the surface of the polyester ranging from 0.1 meq/g to above 3 meq/g, e.g., up to 5 meq/g, or at least 6 meq/g.

In embodiments, the sulfonated polymer is a selectively sulfonated negative-charged anionic block copolymer. The term “selectively sulfonated” definition to include sulfonic acid as well as neutralized sulfonate derivatives. The sulfonate group can be in the form of metal salt, ammonium salt or amine salt.

Depending on the applications and the desired properties, the sulfonated polymer can be modified (or funcationalized). In embodiments, the sulfonated polymer is neutralized with any of various metal counterions, including alkali, alkaline earth, and transition metals, with at least 10% of the sulfonic acid groups being neutralized. In embodiments, the sulfonated polymer is neutralized with inorganic or organic cationic salts, e.g, those based on ammonium, phosphonium, pyridinium, sulfonium and the like. Salts can be monomeric, oligomeric, or polymeric. In embodiments, the sulfonated polymer is neutralized with various primary, secondary, or tertiary amine-containing molecules, with >10% of the sulfonic acid or sulfonate functional groups being neutralized.

In embodiments, the sulfonic acid or sulfonate functional group is modified by reaction with an effective amount of polyoxyalkyleneamine having molecular weights from 140 to 10,000. Amine-containing neutralizing agents can be mono-functional or multi-functional; monomeric, oligomeric, or polymeric. In alternative embodiments, the sulfonated polymer is modified with alternative anionic functionalities, such as phosphonic acid or acrylic and alkyl acrylic acids.

In embodiments, amine containing polymers are used for the modification of the sulfonated polymers, forming members of a class of materials termed coaservates. In examples, the neutralizing agent is a polymeric amine, e.g., polymers containing benzylamine functionality. Examples include homopolymers and copolymers of 4-dimethylaminostyrene which has been described in U.S. Pat. No. 9,849,450, incorporated herein by reference. In embodiments, the neutralizing agents are selected from polymers containing vinylbenzylamine functionality, e.g., polymers synthesized from poly-p-methylstyrene containing block copolymers via a bromination-amination strategy, or by direct anionic polymerization of amine containing styrenic monomers. Examples of amine functionalities for functionalization include but are not limited to p-vinylbenzyldimethylamine (BDMA), p-vinylbenzylpyrrolidine (VBPyr), p-vinylbenzyl-bis(2-methoxyethyl)amine (VBDEM), p-vinylbenzylpiperazine (VBMPip), and p-vinylbenzyldiphenylamine (VBDPA). In embodiments, corresponding phosphorus containing polymers can also be used for the functionalization of the sulfonated polymers.

In embodiments, the monomer or the block containing amine functionality or phosphine functionality can be neutralized with acids or proton donors, creating quaternary ammonium or phosphonium salts. In other embodiments, the sulfonated polymer containing tertiary amine is reacted with alkylhalides to form functional groups, e.g., quaternized salts. In some embodiments, the sulfonated polymer can contain both cationic and anionic functionality to form so-called zwitterionic polymers.

In some embodiments, the sulfonated polymer is a selectively sulfonated negative-charged anionic block copolymer, which “selectively sulfonated” definition to include sulfonic acid as well as neutralized sulfonate derivatives. The sulfonate group can be in the form of metal salt, ammonium salt or amine salt. In embodiments, the sulfonated block polymer has a general configuration A-B-A, (A-B)ₙ(A), (A-B-A)ₙ, (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof; where n is an integer from 0 to 30, or 2 to 20 in embodiments; and X is a coupling agent residue. Each A and D block is a polymer block resistant to sulfonation. Each B block is susceptible to sulfonation. For configurations with multiple A, B or D blocks, the plurality of A blocks, B blocks, or D blocks can be the same or different.

In embodiments, the A blocks are one or more segments selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof. If the A segments are polymers of 1,3-cyclodiene or conjugated dienes, the segments will be hydrogenated subsequent to polymerization of the block copolymer and before sulfonation of the block copolymer. The A blocks may also contain up to 15 mol % of the vinyl aromatic monomers such as those present in the B blocks.

In embodiments, the A block is selected from para-substituted styrene monomers selected from para-methyl styrene, para-ethyl styrene, para-n-propyl styrene, para-iso-propyl styrene, para-n-butyl styrene, para-sec-butyl styrene, para-iso-butylstyrene, para-t-butylstyrene, isomers of para-decylstyrene, isomers of para-dodecylstyrene and mixtures of the above monomers. Examples of para-substituted styrene monomers include para-t-butylstyrene and para-methylstyrene, with para-t-butylstyrene being most preferred. Monomers may be mixtures of monomers, depending on the particular source. In embodiments, the overall purity of the para-substituted styrene monomers be at least 90%-wt., or >95%-wt., or >98%-wt. of the para-substituted styrene monomer.

In embodiments, the block B comprises segments of one or more polymerized vinyl aromatic monomers selected from unsubstituted styrene monomer, ortho-substituted styrene monomers, meta-substituted styrene monomers, alpha-methylstyrene monomer, 1,1-diphenylethylene monomer, 1,2-diphenylethylene monomer, and mixtures thereof. In addition to the monomers and polymers noted, in embodiments the B blocks also comprises a hydrogenated copolymer of such monomer (s) with a conjugated diene selected from 1,3-butadiene, isoprene and mixtures thereof, having a vinyl content of between 20 and 80 mol percent. These copolymers with hydrogenated dienes can be any of random copolymers, tapered copolymers, block copolymers or controlled distribution copolymers. The block B is selectively sulfonated, containing from about 10 to about 100 mol % sulfonic acid or sulfonate salt functional groups based on the number of monomer units. In embodiments, the degree of sulfonation in the B block ranges from 10 to 95 mol %, or 15-80 mol %, or 20-70 mol %, or 25-60 mol %, or >20 mol %, or >50 mol %.

The D block comprises a hydrogenated polymer or copolymer of a conjugated diene selected from isoprene, 1,3-butadiene and mixtures thereof In other examples, the D block is any of an acrylate, a silicone polymer, or a polymer of isobutylene with a number average molecular weight of >1000, or >2000, or >4000, or >6000.

The coupling agent X is selected from coupling agents known in the art, including polyalkenyl coupling agents, dihaloalkanes, silicon halides, siloxanes, multifunctional epoxides, silica compounds, esters of monohydric alcohols with carboxylic acids, (e.g. methylbenzoate and dimethyl adipate) and epoxidized oils.

The antimicrobial and mechanical properties of the sulfonated block copolymer can be varied and controlled by varying the amount of sulfonation, the degree of neutralization of the sulfonic acid groups to the sulfonated salts, as well as controlling the location of the sulfonated group(s) in the polymer. In embodiments and depending on the applications, e.g., one with the need for water dispersity/solubility, or at the other spectrum, one with the need for sufficient durability with constant wiping with water based cleaners, the sulfonated block copolymer can be selectively sulfonated for desired water dispersity properties or mechanical properties, e.g., having the sulfonic acid functional groups attached to the inner blocks or middle blocks, or in the outer blocks of a sulfonated block copolymer, as in U.S. Pat. No. 8,084,546, incorporated by reference. If the outer (hard) blocks are sulfonated, upon exposure to water, hydration of the hard domains may result in plasticization of those domains and softening, allowing dispersion or solubility.

The sulfonated copolymer in embodiments is as disclosed in Patent Publication U.S. Pat. Nos. 9,861,941, 8,263,713, 8,445,631, 8,012,539, 8,377,514, 8,377,515, 7,737,224, 8,383,735, 7,919,565, 8,003,733, 8,058,353, 7,981,970, 8,329,827, 8,084,546, 8,383,735, 10,202,494, and 10,228,168, the relevant portions are incorporated herein by reference.

In embodiments, the sulfonated block copolymer has a general configuration A-B-(B-A)₁₋₅, wherein each A is a non-elastomeric sulfonated monovinyl arene polymer block and each B is a substantially saturated elastomeric alpha-olefin polymer block, said block copolymer being sulfonated to an extent sufficient to provide at least 1% by weight of sulfur in the total polymer and up to one sulfonated constituent for each monovinyl arene unit. The sulfonated polymer can be used in the form of their acid, alkali metal salt, ammonium salt or amine salt.

In embodiments, the sulfonated block copolymer is a sulfonated polystyrene-polyisoprene-polystyrene, sulfonated in the center segment. In embodiments, the sulfonated block copolymer is a sulfonated t-butylstyrene/isoprene random copolymer with C═C sites in their backbone. In embodiments, the sulfonated polymer is a sulfonated SBR (styrene butadiene rubber) as disclosed in U.S. Pat. No. 6,110,616 incorporated by reference. In embodiments, the sulfonated polymer is a water dispersible BAB triblock, with B being a hydrophobic block such as alkyl or (if it is sulfonated, it becomes hydrophilic) poly(t-butyl styrene) and A being a hydrophilic block such as sulfonated poly(vinyl toluene) as disclosed in U.S. Pat. No. 4,505,827 incorporated by reference.

In embodiments, the sulfonated block copolymer is a functionalized, selectively hydrogenated block copolymer having at least one alkenyl arene polymer block A and at least one substantially completely, hydrogenated conjugated diene polymer block B, with substantially all of the sulfonic functional groups grafted to alkenyl arene polymer block A (as disclosed in U.S. 5,516,831, incorporated by reference). In embodiments, the sulfonated polymer is a water-soluble polymer, a sulfonated diblock polymer of t-butyl styrene/styrene, or a sulfonated triblock polymer of t-butyl styrene-styrene-t-butyl styrene as disclosed in U.S. Pat. No. 4,492,785 incorporated by reference. In embodiments, the sulfonated block copolymer is a partially hydrogenated block copolymer.

In embodiments, the sulfonated polymer is a midblock-sulfonated triblock copolymer, or a midblock-sulfonated pentablock copolymer or, e.g., a poly(p-tert-butylstyrene-b-styrenesulfonate-b-p-tert-butylstyrene), or a poly[tert-butylstyrene-b-(ethylene-alt-propylene)-b-(styrenesulfonate)-b-(ethylene-alt-propylene)-b-tert-butylstyrene.

In embodiments, the sulfonated polymer contains >15 mol %, or >25 mol %, or >30 mol %, or >40 mol %, or >60 mol % sulfonic acid or sulfonate salt functional groups based on the number of monomer units in the polymer that are available or susceptible for sulfonation, e.g., the styrene monomers.

In embodiments, the sulfonated polymer has an ion exchange capacity of >0.5 meq/g, or >0.75 meq/g, or >1.0 meq/g, or >1.5 meq/g, or >2.0 meq/g, or >2.5 meq/g, or <5.0 meq/g.

Optional Additives: In embodiments, the sulfonated polymer further contains or can be complexed with, or otherwise form mixtures, compounds, etc. with, tertiary sulfonium, quaternary ammonium and phosphonium containing polymers, chitosan and other naturally occurring antimicrobial polymers, ion-exchange resins, metallic-based micro and nano-structured materials such as silver, copper, zinc and titanium and their oxides, for enhanced antimicrobial effectiveness.

In embodiments, the sulfonated polymer further comprises additives for safety effects, e.g., luminescent additives such as phosphorescent and fluorescence that would help or enable the sulfonated polymer layer to illuminate, or optical brighteners additives that illuminate under a special UV or black light tracer, allowing for physical inspections to verify that intended surfaces are coated or protected with intended sulfonated polymer material for antimicrobial/self-disinfecting effects.

In addition to the above optional components, other additives such as plasticizers, tackifiers, surfactants, film forming additives, dyes, pigments, cross-linkers, UV stabilizers, UV absorbers, catalysts, highly conjugated particles, sheets, or tubes (e.g. carbon black, graphene, carbon nanotubes), etc. may be incorporated in any combination to the extent that they do not reduce the efficacy of the material.

Properties of Sulfonated Polymer: In embodiments, the sulfonated polymer is characterized as being sufficiently sulfonated to have an IEC of >0.5 meq/g, or 1.5-3.5 meq/g, or >1.25 meq/g, or >2.2 meq/g, or >2.5 meq/g, or >4.0 meq/g, or <4.0 meq/g.

In embodiments, the sulfonated polymer is characterized as having a surface pH of <3.0, or <2.5, or <2.25, or <2.0, or <1.80. It is believed that a sufficiently low surface level, as a result of the presence of sulfonic acid functional groups in the protective layer, would have catastrophic effects on microbes that come in contact with the surface.

The sulfonated polymer works effectively in destroying/inactivating >90%, or >95%, or >99%, or >99.5%, or >99.9% of microbes within 120 minutes of exposure, within 60 minutes of exposure, within <30 minutes of exposure, or <5 minutes of exposure or contact with microbes, including but not limited to MRSA, vancomycin-resistant Enterococcus faecium, X-MulV, PI-3, SARS-CoV-2, carbapenem-resistant Acinetobacter baumannii, and influenza A virus. In embodiments with polymer containing quaternary ammonium group, the material is effective in killing target microbes including Staphylococcus aureus, Escherichia coli, Staphylococcus albus, Escherichia coli, Rhizoctonia solani, and Fusarium oxysporum. The sulfonated polymer remains effective in killing microbes even after 4 hours, or after 12 hours, or at least 24 hours, or for at least 48 hours. In embodiments, the sulfonated polymer remains effective in killing microbes for at least 3 months, or for at least 6 months.

Air Filter Applications: Pollen particles are about 10 μm or bigger. Bacteria are often about 1 μm. Research shows that the particle size of SARS-CoV-2, the virus that causes COVID-19, is around 0.1 μm. These viral particles are human-generated, so the virus is trapped in respiratory droplets and droplet nuclei (dried respiratory droplets) that are larger than an individual virus. Most of the respiratory droplets and particles exhaled during talking, singing, breathing, and coughing are less than 5 μm in size. These particles or droplets are containing the viral particles are killed once they come into contact with sulfonated polymer surface.

It should be noted that very small particles, e.g., even as small as 0.01 μm, or those that become aerosolized can be captured due to a natural phenomenon called Brownian motion, i.e., when particles have so little mass than they actually bounce around and move in random zigzag pattern. In embodiments, the sterilizing efficacy of sulfonated polymer in filter media increases with decreasing particle size, i.e., with microbes, e.g., viruses of extremely small size even for filter media that are sufficiently of larger sizes, or not sufficiently dense to capture the virus on the surface. As the virus bounces around in Brownian motion and comes into contact with the sulfonated polymer surface, it is deactivated or killed upon contact. Additionally, as air in buildings, e.g., a home, a facility, a vehicle, etc., is recirculated, the same air will pass through the filter media multiple times in a day. After several rounds after passing through the filter media comprising the sulfonated polymer, the air will get cleaned.

In embodiments, for effective capture of microbes, particularly microbes trapped in droplets larger than individual viruses, with highly efficient ventilation without detrimental effects on overall air filter system performance, the sulfonated polymer is for use with air filters having a minimum filtration efficiency target of MERV 13, for residential or commercial air filter systems with. A MERV 13 filter is at least 50% efficient at capturing particles in the 0.3 μm to 1.0 μm size range and 85% efficient at capturing particles in the 1 μm to 3 μm size range. In embodiments, the sulfonated polymer is for use with a MERV 14 filter, for >75% and 90% efficient, respectively, at capturing those same particles. A MERV 8 filter is 85% efficient at capturing particles in the 3.0 to 10 μm size range.

In embodiments to effectively and/or immediately sterilize the air, the sulfonated polymer is for use with HEPA filters with 99.97% efficiency in capturing particles 0.3 μm in size, for home use as portable air purifiers, or for use in medical facilities, e.g., hospitals, health clinics, medical testing locations, workout rooms, or public waiting areas.

Although the sulfonated polymer is particularly suitable for use in air filter applications for filtering air in buildings, the air filter employing sulfonated polymer, e.g., a sulfonated block copolymer, can be particularly useful in microfilters having a pore size sufficient for removing and/or sterilizing bacteria and other minute particulates, for a residential or commercial refrigerator. The microfilter can function in conjunction with the refrigerator's integral fan to rid the refrigerator air of airborne particulates illustrated by, but not limited to, mold spores, bacteria, and viruses. In embodiments, the sulfonated polymer is for used with air filters employed in cabins of air planes or vehicles, e.g., buses, automobiles, trains, etc., for the filtering of air entering the cabins.

Depending on the filter type, whether to meet the filtration standards of HEPA filters, ULPA filters, or MERV filters, the end-use applications such as air ducts, furnace, air conditioners, refrigerators, air purifiers, etc., the filter media can be of different materials of construction with pores/capillaries for air to flow through. Filter media can also be of different designs, e.g., depth filtration media or surface filtration media. The sulfonated block copolymer can be used for the protection of either depth filtration media or surface filtration media.

For surface filtration, the particles are collected and accumulate on the surface of the filter medium. Surface filters are typically made from microporous membranes, providing the most effective filtration efficiency having a membrane structure with millions of pores to capture submicron particles. Generally, the backing substrate of surface filtration membrane may merely be a support that plays a minimal part in the filtration process, with particulates being collected on the membrane surface with no penetration of the particulate into or beyond the filter media.

For depth filtration, the particles tend to penetrate the filter medium and become captured throughout the depth of the media. Depth filtration media has a broad pore size distribution and relies on adsorption-retention for a portion of its dirt holding capacity. Depth filters are made from coarse fibers such as meltblown nonwovens that are much more open than surface filters. Depth filtration media are more suitable for removing a broader range of particles with a maximal dimension of greater than 10 μm. Examples of depth filtration media include spunbond and meltblown fabrics.

In embodiments, the filter media is supported or housed in a cartridge that can be taken in and out of an air filter device, e.g., an air purifier, for replacement. The filter media in embodiments is supported by a frame or with a backing, pre-sized to fit a particular furnace or air conditioning system. In other embodiments, the filter medium is freestanding and self-supported, which can be cut to size for use as an air filter itself. In embodiments, the filter medium is enclosed in a housing, e.g., a vacuum cleaner bag comprising porous paper or nonwoven web.

In embodiments, the filter medium comprises a pile of woven, felted, or nonwoven fabric, or filaments, or fibers such as microfibers, nanofibers, etc., laid together or weaved into a mat for the filtering of particles, with a low porosity (“sieve size”) rating. The sieve size rating is the basis for the filtering of particles from the air stream. In general, the smaller the fiber diameter, the smaller the filter media sieve size. In embodiments, the filter medium comprises a mat of individual fibers made from materials such as fiberglass, metals such as stainless steel, and polymers, oriented randomly and perpendicular to air flow. In embodiments, the filter medium comprises polymeric expanded foam.

In embodiments, the filter medium is protected by a pre-filter, e.g., a dust layer, to remove larger particles from the incoming airstream.

In embodiments, the filter medium is a composite medium having different “tiers” or stacked filters of different designs (e.g., foam filter, fiber filter, etc.), different materials (e.g., different polymers, different fibers, etc.), different sieve sizes, bonded together as one structure, or as separate layers, forming a stratified structure for filtering particles of different sizes.

When in use, the filter medium inlet side with air to be “sterilized” is “upstream,” and the filter medium discharge side is “downstream”—after the air has been treated with the microbes having been effectively killed by the self-sterilizing sulfonated polymer. The filter medium is air permeable. In embodiments, the filter medium consists of openings or air passages that are defined by the structural elements of the medium, e.g., the fibers forming the medium. In embodiments, the filter medium has a plurality of capillaries or air passages extending from one side of the medium (upstream) to the other side (downstream), with pores as individual restrictions within the capillaries.

Methods for Incorporating Sulfonated Polymers into Air Filters: The sulfonated polymer can be incorporated into the air filter in various ways to provide the filter medium with antimicrobial or self-sterilizing effect. In embodiments, at least a surface of the filter medium is coated or protected with the sulfonated polymer. In some embodiments, at least a surface of the filter medium housing, e.g., filter vacuum bag, is coated or protected with the sulfonated polymer. In other embodiments, the sulfonated polymer is used as material of construction for at least a portion of the filter medium, e.g., the inlet side of the filter media in contact with the air to be filtered.

In embodiments, the sulfonated polymer is first electrospun (e-spun), generating nanoscale to microscale fibers with disinfecting properties. In electrospinning, a solution comprising the sulfonated polymer is fed to a multi-nozzle device or a nozzle free device, and a high voltage is applied. The solution is transformed under the influence of the high voltage into charged jet streams and deposited onto a substrate, or take by a collector. The polymer in the jet stream solidifies thereby forming nanofibers or microfibers. Electrospinning can be used to generate sulfonated microfibers or nanofibers of less than 400 nm, or less than 200 nm, or 50-300 nm, or less than 250 microns, or 50-150 microns, or 40-90 microns, depending on the specific electrospinning conditions employed.

In embodiments, the sulfonated polymer in solution is e-spun via a multi-nozzle device or a nozzle-free electrospinning with the use of nozzle free device onto a filter media substrate (e.g., a mat) as laid out on a moving conveyer belt. In embodiments, the amount of sulfonated polymers (or density) of e-spun sulfonated block copolymer on the filter media ranges from 1-30 grams per square meter per mil thickness, or 2-10 g/m², or at least 3 g/m², or less than 5 g/m², for an electrospun sulfonated polymer mat having a thickness of <500 nm, <200 nm, or <100 nm, or >50 nm, or 25-600 nm, or 40-400 nm.

By controlling factors including but not limited to the concentration of the sulfonate polymer in solution, the diameter of the nozzle, the speed of the melt spinning process, the speed the conveyor belt, the spinning distance, and the applied voltage, the thickness and amount of electrospun sulfonated nanofibers or microfibers can be controlled to provide sufficient coverage of the surface of the filter medium pores and air capillaries, without plugging them. The filter media substrate can be subsequently cut into sizes for packaging/sold to be used as air filters.

In embodiments, the electrospun sulfonated microfibers or nanofibers form a stand-alone nanofiber or microfiber layer or mat, having a thickness of <500 nm, <200 nm, or <100 nm, or <50 nm, or 25-600 nm, or 40-400 nm, for use with the filter medium and supported by the filter medium substrate made of a different material, e.g., e-spun poly(tetrafluoroethylene), etc. Multiple nanofiber layers can be used, sandwiched or alternating with layers of other materials, forming the filter medium. In embodiments, the stand-alone e-spun sulfonated polymer mat is used in conjunction with other polymeric layers (as support or substrate) forming the filter medium.

In embodiments, the electrospun sulfonated microfibers or nanofibers are interweaved with other fibers, e.g., microfibers, submicron fibers and nanofibers from other different polymers, forming the filter medium. Depending on the sulfonated polymer employed, in embodiments in addition to self-sterilizing properties, the polymer also facilities or controls the vapor transmission rate and provides an environment that would accelerate wound healing.

Examples of other different polymers for use in the filter medium include but are not limited to cellulose acetate (CA), polyolefin, polyamide 6 (PA 6), polystyrene (PS), polyacrylonitrile (PAN), polyvinylpyrrolidone (PVP), polyvinyl alcohol (PVA), polyethylene oxide (PEO), poly(lactic acid) (PLA), poly(lactic-co-glycolic acid) (PLGA), polybutylene terephthalate (PBT) and polyurethane (PU), or natural polymers such as gelatin, chitosan and polyhydroxybutyrate-co-hydroxyvalerate (PHBV).

The sulfonated polymer may be applied onto the surface of the filter medium by dissolving the polymer into a suitable solvent and then applying the sulfonated polymer onto the filter medium as a coating by using methods including but not limited to spray coating, dipping, and painting onto the surface of the filter medium. The sulfonated polymer is applied such that at least a portion of the surface of the air passages (or the pores), e.g., at least 5%, or at least 10%, or at least 15%, or at least 20%, is coated with a thin layer, <100 μm, or <10 μm, or <5 μm, or <1 μm of sulfonated polymer, such that microbes, microbe-containing particles (droplets) are effectively killed upon contact upon being drawn into the filter medium.

In embodiments, the sulfonated polymer is first applied onto the fibers (by any of spray coating, dipping, etc.), then the sulfonated polymer coated fiber is weaved or matted forming the filter media, or at least a portion of the filter medium inlet side.

Example 1: Tests were conducted to evaluate antimicrobial efficacy & the long-lasting antiviral properties of sulfonated polymers, film samples of sulfonated penta block copolymer (SPBC) of the structure poly[tert-butylstyrene-b-(ethylene-alt-propylene)-b-(styrene-co-styrene-sulfonate)-b-(ethylene-alt-propylene)-tert-butylstyrene] with 52% sulfonation were cast out of 1:1 mixture of toluene and 1-propanol. The sulfonated polymer film samples were subjected to abrasion testing of 2200 cycles in the presence of 3 common disinfectants: 1) 70% ethanol, benzalkonium chloride, and quaternary ammonia], and exposure to SARS-CoV-2 virus suspension of concentration 10⁷ pfu/ml.

After 2 hours of contact, viable virus was recovered from each sample by washing twice with 500 μl of DMEM tissue culture media containing 10% serum, and measured by serial dilution plaque assay. Gibco Dulbecco's Modified Eagle Medium (DMEM) is a basal medium for supporting the growth of many different mammalian cells. The results demonstrate that, after abrasion testing representing approximately one year of cleaning (6 disinfectant wipes/day), surface pro Gibco Dulbecco's Modified Eagle Medium (DMEM) is a widely used basal medium for supporting the growth of many different mammalian cells.

Example 2: Woven fabric of nylon 6, 6 fibers is immersed for 5 minutes in a solution of 0.5 g potassium t-butoxide and 0.5 g methanol in 10 ml of DMSO to provide deprotonated amines on the amide nitrogen in the polymer backbone. The deprotonated polymer is immersed in a solution of 0.33 g of sodium 4-bromobenzylsulfonic acid in 3.3. g DMSO (52° C.) to provide a fabric of polyamide fibers having benzylsulfonate groups attached to the surface thereof. The fabric of sulfonated polyamide fiber is washed with deionized (DI) water and dried to for use in making filter medium.

Example 3: A sulfonated polyester fabric is prepared, for use in making face masks, protective clothing, and the like. First a polyester taffeta made of polyester fiber is put into an acid-resistant sealable container. Sulfuric anhydride previously diluted 10 times with nitrogen gas is brought into contact with the polyester cloth for a sulfonated polyester material. The cloth is then washed with water and dried to produce a sulfonated polyester fabric, which can be subsequently used to make filter medium.

Example 4: The example was conducted to evaluate the effectiveness in inhibiting Aspergillus niger black mold according to the AATCC Test Method 30-2004 Test III. Six different sulfonated block copolymer membrane samples comprising a poly[tert-butylstyrene-b-(ethylene-alt-propylene)-b-(styrenesulfonate)-b-(ethylene-alt-propylene)-b-tert-butylstyrene], at different levels of sulfonation from 26 to 52% were used for the tests. Aspergillus niger, ATCC#6275, was harvested into sterile distilled water containing glass beads. The flask was shaken to bring the spores into suspension. The suspension was used as the test inoculum. One (1.0) mL of the inoculum was even distributed over the surface of Mineral Salts Agar plates. The membrane samples were placed onto the inoculated agar surface. After placement, 0.2 mL of the inoculum was distributed over the surface of each disc. A viability plate of the spore suspension was prepared on Mineral Salts Agar with 3% glucose. A positive growth control was prepared using an untreated cotton duck fabric on Mineral Salts Agar and set up in the same manner as the test items. All samples were incubated at 28° C.±1° C. for 14 days.

The viability plate had acceptable fungal growth as expected confirming the viability of the inoculum. The sample with 26% sulfonation showed microscopic growth on 10% of the sample surface. The other 5 test samples showed no growth, or microscopic growth on 1% of the surface. The control sample showed macroscopic growth on 100% of the surface

As used herein, the term “comprising” means including elements or steps that are identified following that term, but any such elements or steps are not exhaustive, and an embodiment can include other elements or steps. Although the terms “comprising” and “including” have been used herein to describe various aspects, the terms “consisting essentially of” and “consisting of” can be used in place of “comprising” and “including” to provide for more specific aspects of the disclosure and are also disclosed.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items.

Unless otherwise specified, all technical and scientific terms used herein have the same meanings as commonly understood by one of skill in the art to which the disclosed disclosure belongs. the recitation of a genus of elements, materials or other components, from which an individual component or mixture of components can be selected, is intended to include all possible sub-generic combinations of the listed components and mixtures thereof.

The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. To an extent not inconsistent herewith, all citations referred to herein are hereby incorporated by reference.

1. An air filter device comprising:
a filter medium having an inlet surface for taking in air, and a discharge surface for filtered air, the filter medium having a plurality of air passages for the air to flow therethrough;
wherein at least a portion of surface of the air passages is coated with a sulfonated polymer layer for killing at least 95% microbes in the air within 30 minutes of contact with the sulfonated polymer; and
wherein the sulfonated polymer layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof, the sulfonated polymer has a degree of sulfonation of at least 10%.

2. The air filter of claim 1, wherein the sulfonated polymer has an ionic exchange capacity (IEC) of >0.5 meq/g.

3. The air filter of claim 1, wherein the sulfonated polymer is selectively sulfonated to contain from 10-100 mol % sulfonic acid or sulfonate salt functional groups based on the number of monomer units or blocks in the sulfonated polymer susceptible to sulfonation, for the coating material to kill at least 95% of microbes within 30 minutes of contact.

4. The air filter of claim 1, wherein the sulfonated polymer is a selectively sulfonated negative-charged anionic block copolymer having a general configuration of: A-B-A, (A-B)n(A), (A-B-A)n, (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof, wherein
n is an integer from 0 to 30,
X is a coupling agent residue,
each A and D block is a polymer block resistant to sulfonation,
each B block is susceptible to sulfonation,
the A block is selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof;
the B block is a vinyl aromatic monomer, and
the D block is a hydrogenated polymer or copolymer of a conjugated diene selected from isoprene, 1,3-butadiene and mixtures thereof; and
wherein the block B is selectively sulfonated to contain from 10-100 mol % sulfonic acid or sulfonate salt functional groups based on the number of monomer units, for the coating material to kill at least 99% of microbes within 30 minutes of contact.

5. The air filter of claim 1, wherein the sulfonated polymer layer has a surface pH of <3.0.

6. The air filter of claim 1, wherein the sulfonated polymer is neutralized with at least a salt selected from ammonium, phosphonium, pyridinium, and sulfonium salts.

7. The air filter of claim 1, wherein the sulfonated polymer is a selectively sulfonated negative-charged anionic block copolymer, having at least one alkenyl arene polymer block A and at least one substantially completely, hydrogenated conjugated diene polymer block B, with substantially all of the sulfonic functional groups grafted to alkenyl arene polymer block A for the block A to be a hydrophilic end-block.

8. The air filter of claim 1, wherein the filter medium has a MERV rating of 8 to 14.

9. The air filter of claim 1, wherein the filter medium is a HEPA filter medium.

10. The air filter of claim 1, wherein the filter medium comprises any of woven, felted, nonwoven fabric, filaments, microfibers, nanofibers laid or weaved together as a mat.

11. The air filter of claim 1, wherein the filter medium comprises electrospun sulfonated polymer on a support structure comprising electrospun poly(tetrafluoroethylene).

12. The air filter of claim 1, wherein the filter medium comprises a mat of individual fibers made from any of fiberglass, stainless steel, and polymers, wherein the fibers are oriented randomly and perpendicular to air flow.

13. The air filter of claim 1, wherein the filter medium comprises polymeric expanded foam.

14. The air filter of claim 1, wherein at least 10% of the surface of the air passages is coated with the sulfonated polymer layer for a thickness of <100 μm.

15. A method for the preparation of an anti-microbial filter device, comprising:
providing a solution comprising sulfonated polymer and a solvent;
feeding the sulfonated polymer in solution to a multi-nozzle device or a nozzle-free electrospinning with nozzle free device under influence of a high voltage, generating charged jet streams of sulfonated polymer in solution;
depositing the charged jet streams of sulfonated polymer in solution onto a filter mat comprising a plurality of fine fibers for air permeation, wherein the plurality of fine fibers are coated with the sulfonated polymer at a rate from 1-30 grams sulfonated polymer per square meter per mil thickness of the filter mat, forming a self-sterilizing filter medium, for killing at least 95% microbes in the air within 30 minutes of contact with the filter medium;
wherein the sulfonated polymer layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof, the sulfonated polymer has a degree of sulfonation of at least 10%.
The air filter of claim 1, wherein the sulfonated polymer has an ionic exchange capacity (IEC) of >0.5 meq/g.

16. The method of claim 15, wherein the sulfonated polymer is selectively sulfonated to contain from 10-100 mol % sulfonic acid or sulfonate salt functional groups based on the number of monomer units or blocks in the sulfonated polymer susceptible to sulfonation, for the coating material to kill at least 95% of microbes within 30 minutes of contact.

17. The method of claim 15, wherein the sulfonated polymer is a selectively sulfonated negative-charged anionic block copolymer having a general configuration of: A-B-A, (A-B)n(A), (A-B-A)n, (A-B-A)ₙX, (A-B)nX, A-D-B, A-B-D, A-D-B-D-A, A-B-D-B-A, (A-D-B)ₙA, (A-B-D)ₙA (A-D-B)ₙX, (A-B-D)ₙX or mixtures thereof, wherein
n is an integer from 0 to 30,
X is a coupling agent residue,
each A and D block is a polymer block resistant to sulfonation,
each B block is susceptible to sulfonation,
the A block is selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof;
the B block is a vinyl aromatic monomer, and
the D block is a hydrogenated polymer or copolymer of a conjugated diene selected from isoprene, 1,3-butadiene and mixtures thereof; and
wherein the block B is selectively sulfonated to contain from 10-100 mol % sulfonic acid or sulfonate salt functional groups based on the number of monomer units, for the coating material to kill at least 99% of microbes within 30 minutes of contact.

18. The method of claim 15, wherein the sulfonated polymer layer has a surface pH of <3.0.

19. The method of claim 15, wherein the sulfonated polymer is neutralized with at least a salt selected from ammonium, phosphonium, pyridinium, and sulfonium salts.

20. The method of claim 15, wherein the sulfonated polymer is a selectively sulfonated negative-charged anionic block copolymer, having at least one alkenyl arene polymer block A and at least one substantially completely, hydrogenated conjugated diene polymer block B, with substantially all of the sulfonic functional groups grafted to alkenyl arene polymer block A for the block A to be a hydrophilic end-block.
